(19) United States
(12) Patent Application Publication
(10) Pub. No.: US 2015/0087928 A1
(43) Pub. Date: Mar. 26, 2015

(54) MEASURING TISSUE VOLUME WITH DYNAMIC AUTORECONFIGURATION

(71) Applicant: Leo Technologies, Inc., Boulder, CO (US)

(72) Inventors: Ronald Podhajsky, Boulder, CO (US); Arlen J. Reschke, Longmont, CO (US)

(21) Appl. No.: 14/491,836

(22) Filed: Sep. 19, 2014

Related U.S. Application Data
(60) Provisional application No. 62/027,079, filed on Jul. 21, 2014.
(60) Provisional application No. 61/943,997, filed on Feb. 24, 2014.
(60) Provisional application No. 61/880,872, filed on Sep. 21, 2013.
(60) Provisional application No. 61/880,868, filed on Sep. 21, 2013.

(51) Int. Cl. A61B 5/00; A61B 5/01; A61B 5/02

(52) U.S. Cl. CPC A61B 5/443; A61B 5/0059; A61B 5/02; A61B 5/01
USPC 600/301; 600/476; 600/479

(57) ABSTRACT

Implementations disclosed herein provide a hydration monitoring technology. In one implementation, a hydration monitoring device measures whole body hydration levels by analysis of changes in vascular volume caused by pulsatile pressure waves and in tissue volume in response to the pulsatile pressure. The hydration monitoring device uses a light-based measurement technique to transmits light, reflectively or transmissively, through tissue. Based on wavelength measurements of the detected light, the hydration monitoring device produces a PPG waveform representing characteristic effects of hydration. The hydration monitoring device monitors operating condition signals associated with the hydration monitoring device. By monitoring operating condition signals, the hydration monitoring device determines whether the operating condition signals satisfy an analysis condition, and modifies the optical sensing operations. Based on the modified optical sensing operations, the PPG waveform is analyzed, and the hydration monitoring device determines a hydration metric representative of hydration levels in the body of the subject.

### CROSS REFERENCE TO RELATED APPLICATIONS

The present application claims priority to pending U.S. Provisional Patent Application Ser. No. 61/880,868, entitled “System and Method for Monitoring Body Hydration Levels with a Non-Obtrusive Form Factor,” filed on Sep. 21, 2013, U.S. Provisional Patent Application Ser. No. 61/880,872, entitled “System and Method for Non-Invasive Plethysmogram Measurement,” filed on Sep. 21, 2013, U.S. Provisional Patent Application No. 61/943,997, entitled “Algorithm that Derives Hydration Levels From a Plethysmogram,” filed on Feb. 24, 2014, and U.S. Provisional Patent Application Ser. No. 62/027,079, entitled “Hydration Monitoring,” filed on Jul. 21, 2014, all of which are specifically incorporated by reference for all they disclose and teach.

The present application is related to U.S. patent application Ser. No. ______ [Docket No. 277003USP1], entitled “Data Integrity,” U.S. patent application Ser. No. ______ [Docket No. 277002USP1], entitled “Hydration Monitoring,” and U.S. patent application Ser. No. ______[Docket No. 277003USP2], entitled “Dynamic Profiles,” all of which are filed concurrently herewith, and specifically incorporated by reference for all they disclose and teach.

### BACKGROUND

Physiological characteristics in the body, including hydration, can be measured by a variety of techniques, such as skin electrical impedance or optical spectroscopic techniques. Optical spectroscopic techniques may include detecting a photoplethysmographic (PPG) waveform using optical transmitters and optical sensors. In some implementations, PPG signals measure local blood pressure changes in a user's extremity or by ventilation. These waveform measurements can then be analyzed for assessing certain biological conditions.

### SUMMARY

Implementations disclosed herein provide a hydration monitoring technology, although other biometrics may also be determined using or in combination with other similar techniques. In one implementation, a hydration monitoring system measures whole body hydration levels by analysis of changes in vascular volume caused by pulsatile pressure waves and in tissue volume in response to the pulsatile pressure. The hydration monitoring system includes a hydration monitoring device, which uses a light-based measurement technique to measure hydration levels and heart rate during different activities and at rest. In one implementation, a light source operatively connected to a light sensor, transmits light, reflectively or transmissively, through body tissue of a subject. The light sensor detects absorption of the light. Based on wavelength measurements of the detected light, the hydration monitoring device generates a PPG waveform representing characteristic effects of hydration.

In one implementation, the hydration monitoring device monitors operating condition signals associated with the hydration monitoring device. By monitoring operating condition signals, the hydration monitoring device determines whether the operating condition signals satisfy an analysis condition, and modifies the optical sensing operations. Based on the modified optical sensing operations, the PPG waveform is analyzed, and the hydration monitoring device determines a hydration metric representative of hydration levels in the body of the subject.

This Summary introduces a selection of concepts in a simplified form that are further described below in the Detailed Description. This Summary is not intended to identify key features or essential features of the claimed subject matter, nor is it intended to be used to limit the scope of the claimed subject matter. Other feature, details, utilities, and advantages of the claimed subject matter will be apparent from the following more particular Detailed Description of various implementations as further illustrated in the accompanying drawings and defined in the appended claims.

### BRIEF DESCRIPTIONS OF THE DRAWINGS

FIG. 1a illustrates an example hydration monitoring system.

FIG. 1b illustrates a second example hydration monitoring system.

FIG. 1c illustrates a third example hydration monitoring system.

FIG. 2 illustrates a fourth example hydration monitoring system.

FIG. 3 illustrates a block diagram of an example hydration monitoring system circuitry.

FIG. 4 illustrates an example plethysmograph in a hydration monitoring system.

FIG. 5 illustrates example operations for determining a hydration metric.

FIG. 6 illustrates example operations for autoconfiguration of a hydration monitoring system.

FIG. 7 illustrates a block diagram of a computer system in a hydration monitoring system.

### DETAILED DESCRIPTION

Devices, methods, and software using sensors and light sources may be used to produce PPG waveform measurement of hydration levels and heart rate during different activities and at rest. The disclosed technology provides whole body hydration levels by optically measuring changes in vascular volume caused by pulsatile pressure waves and responses by proximal tissue to the pulsatile pressure. Such measurements for whole body hydration levels can be made at a test region (e.g., a wrist). A hybrid of systemic and local hydration monitoring is achieved by measuring both vascular volume and tissue biomechanics that produces more accurate results, which can be communicated in a hydration metric.

In addition to hydration, in other implementations, the disclosed technology also monitors or refines results of monitoring other physiological parameters, including, but not limited to, blood pressure, heart contractility hydration, heart rate, heart contractility, valve performance, vascular compliance, baroreceptor engagement, systemic neural response, local neural response, vascular branch reflections, blood density, vascular pathology, valve pathology, heart pathology, and compensatory reserve index. The data of these other physiological parameters may be used to compute a biometric pursuant to the technology disclosed herein.

To calculate either a hydration metric or other biometric related data, a hybrid of changes in vascular and tissue pressures and/or volumes are analyzed using a light-based measurement technique. In one implementation, the system includes a processor in operative communication with an optical sensor or light sensor and a light source. The light source exposes tissue to light. Light can be reflected through the tissue, or the light can be transmitted through the tissue. The light sensor is configured to detect changes of light absorption through the body tissue to measure changes in body tissue volume in combination of changes in vascular volume within a test region of the body of a subject.

Absorption of a specific wavelength of light energy is dependent on the amount of oxygenated blood in the vessels. Since the heart is a pulsatile pump, blood enters the arteries intermittently with each heartbeat increasing vascular volume and/or pressure. Vessels expand and contract, in response to the changing pressure in the vessels. At the same time, pressure is also dependent on surrounding tissue, which may comprise as much as 60-80% water. When the vessels expand and relax, the amount of blood volume in the observed tissue increases and decreases. The compliance ability to distend and increase volume by pressure of the vessels changes in rhythm with the heartbeat. As overall tissue hydration increases, the compliance of the vessels, both centrally and peripherally, is reduced, and there is more resistance to pressure in the vasculature.

The light absorption in the tissue has a pulsatile component that varies in rhythm with the heartbeat. As the heart beats, the volume of blood increases and travels as a pressure wave through the circulatory system. As blood volume increases in the arteries, the received light intensity reduces. As blood volume in the arteries decreases, the light transmission increases.

A processor, operatively connected to the light sensor, processes the light changes in time variant signals (intensity vs. time) detected by a light sensor. The time variant signals can be amplified to generate an electrical representation in a measureable PPG waveform.

In another implementation, the plethysmographic waveform is measureable by non-optical means. For example, electrical impedance plethysmography also provides a waveform representing the changes in tissue volume and in vascular volume. For either optical or non-optical plethysmographic waveform generation, the measurement and computations of the disclosed technology remain the same.

The waveform provided by the photodetector may be inverted. If the waveform is inverted, the peak of the waveform corresponds to the maximum absorption of the light when the blood vessels are pulsing at their maximum dilation. The lowest part of the peak is the point between heartbeats where there is the minimum dilation of the vessels and less absorption of the light. The PPG waveform represents volume and pressure changes in the circulatory system indicative of characteristic effects of hydration.

Areas of the PPG waveforms are computed that represent the volume and pressure changes in the body. The first area of the PPG waveform, indicative of changes in tissue volume, is referred to as the “Tissue Pressure Area” or “TPA.” A second area of the PPG waveform, indicative of the changes in vascular volume, is referred to as the “Vessel Pressure Area” or “VPA.”

Based on these computations of areas, a hydration metric can be computed based on the different ratios of determined changes in vascular volume and tissue volume in the body. For example, during times of exercise or following exertion, the ratio of the TPA to the VPA provides a hydration metric:

$$HydrationIndex = \frac{TissuePressueArea}{VesselPressureArea}$$

Alternatively, during extended times of rest, the ratio of the VPA to the TPA provides a hydration metric:

$$HydrationIndex = \frac{VesselPressureArea}{TissuePressueArea}$$

These ratios can be inverted and can vary subject to change depending on a variety of factors, including the level of activity (e.g., rest, during exercise, following exertion), physiological conditions, environmental conditions, particular user profile parameters, the specific hydration monitoring device used, or calibration of the hydration monitoring system.

For example, in one implementation, during rest, the hydration index can be computed correlating to a ratio of the TPA to the VPA. Or, in another implementation, during exercise, the hydration index can be computed correlating to a ratio of the VPA to the TPA. Or in another implementation, a particular user profile may trigger a change in taking the ratio of the VPA to the TPA, to taking a ratio of the TPA of the VPA.

In another implementation, multipliers or constants may be used to calculate the hydration metric with the ratio of the TPA to the VPA or the ratio of the VPA to the TPA. Such modification of the ratio can result in better aggregate data. These multipliers or constants can also be implemented as part of a user profile.

Once computed, the hydration monitoring system communicates the hydration metric for presentation via a user interface.

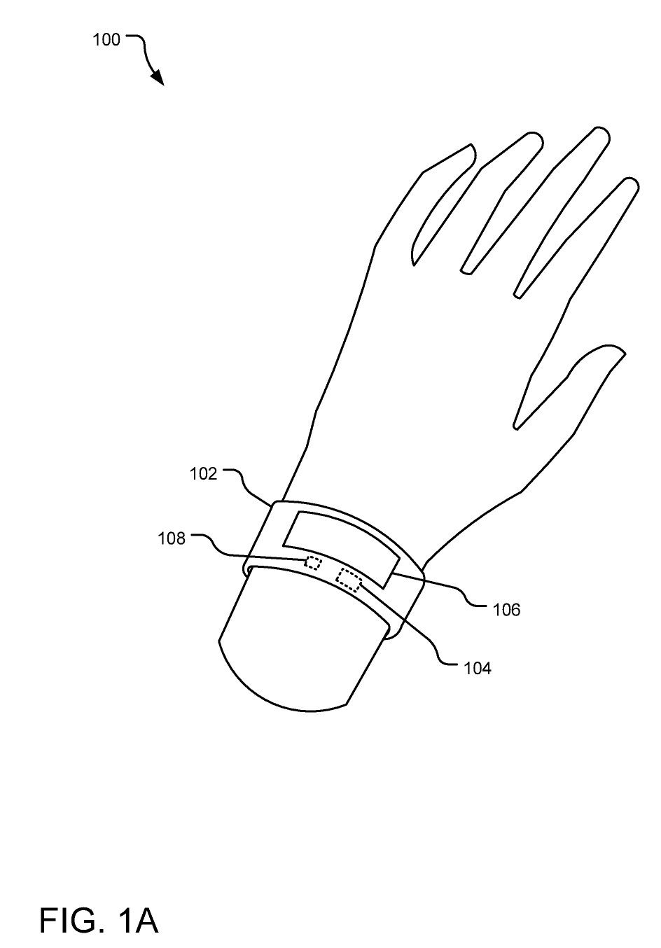

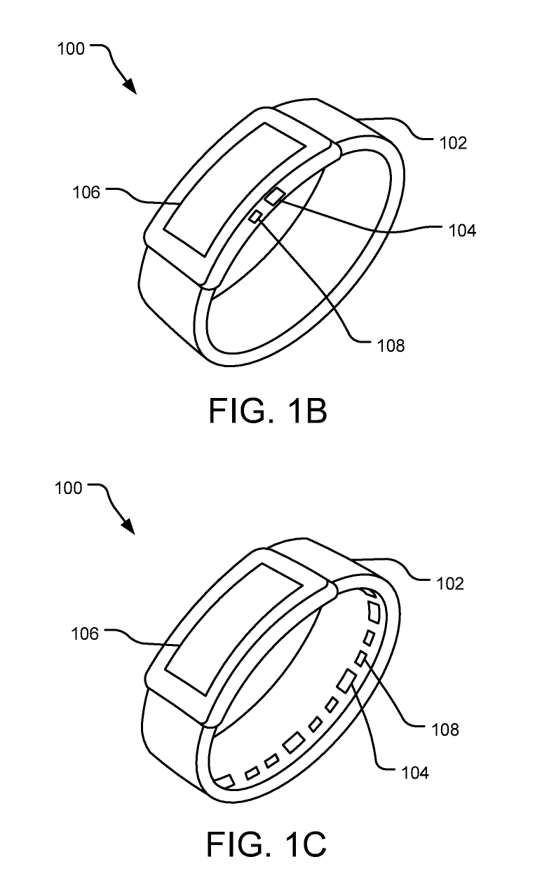

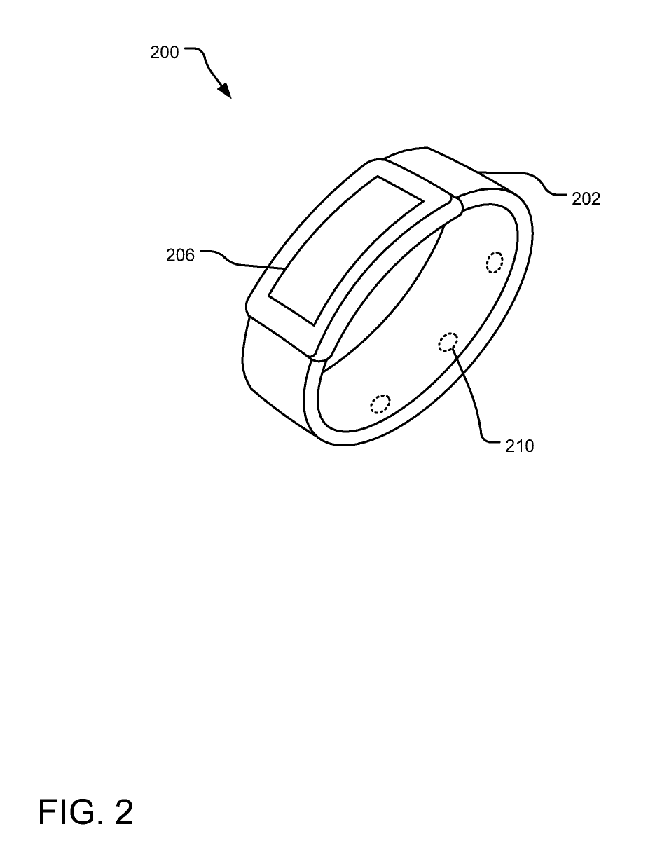

In FIG. 1a, an example hydration monitoring system 100 in the disclosed technology is shown. The system 100 includes sensor circuitry (described further in FIG. 2) configured to acquire and reflectively measure a PPG waveform. The sensor circuitry may be located in a device or monitor, such as a wrist-worn form factor (e.g., watch or wristlet 102), as shown in FIG. 1a. Other implementations may include transmissive PPG measurement systems worn on the fingertip, earlobe, etc., or reflective PPG systems worn on the forehead, fingertip, or other body locations.

Other implementations may include a PPG waveform sensor module that may be incorporated into expandable bandages, clothing (e.g. sweatbands, gloves, sports bras, and other sportswear), sports equipment (e.g., a bike helmet), ear buds, or an anklet. Additional implementations may include the sensor module incorporated into an accessory housing or protective cover used with smart phones, tablets, GPS, and other similar devices. In another implementation, the sensor may be incorporated into a switch button used on a monitoring device or may be incorporated as a biometric contact button exclusively for biometric data readings. In another implementation, monitoring may be facilitated through the device itself, a monitoring service, a computer, wirelessly, or via a medical testing unit.

The PPG waveform sensor module may also be incorporated as a biometric button, such as a finger or a palm contact location. The module may also be incorporated into health and fitness equipment, such as treadmills, elliptical trainers, bicycle handlebars, water bottles, and other similar equipment.

Referring to FIG. 1a, the wristlet 102 has a light detector or light sensor 104 and a light source 108. The light sensor 104 and the light source 108 can be configured to rest on or next to the skin surface in close proximity to the arterial or arteriole vascular components that produce a PPG wave. As further described in detail in FIG. 2, the light source 108 generates light through skin and tissue, and the light is detected by the light sensor 104. A processing unit in the wristlet 102 (or accessible to the wristlet 102) processes the light into analytical PPG pulse data samples, which are then processed into hydration metric data results. The hydration metric data results are displayed on an interface or display 106.

The light sensor 104 and the light source 108 can be located in various configurations and locations in the hydration monitoring system 100. In FIG. 1a, a light sensor 104 is located on the inside of the wristlet 102, adjacent to the user's skin. In another implementation, as shown in FIG. 1b, the light sensor 104 and the light source 108 may be located on the side of the wristlet 102. In this example, a user can wear the wristlet on one wrist, and use the wristlet for measurement in the other wrist or finger on the other arm. In another implementation (not shown), a sensor could be on the top of a wristlet 102, wherein the sensor detects hydration in a person other than the person wearing the wristlet 102 (e.g., a patient uses a first responder's watch to read their hydration). In another implementation (not shown), a wristlet may have a light sensor positioned on one side of the wristlet aimed into the wrist, and another light sensor may be located on another side of the wristlet.

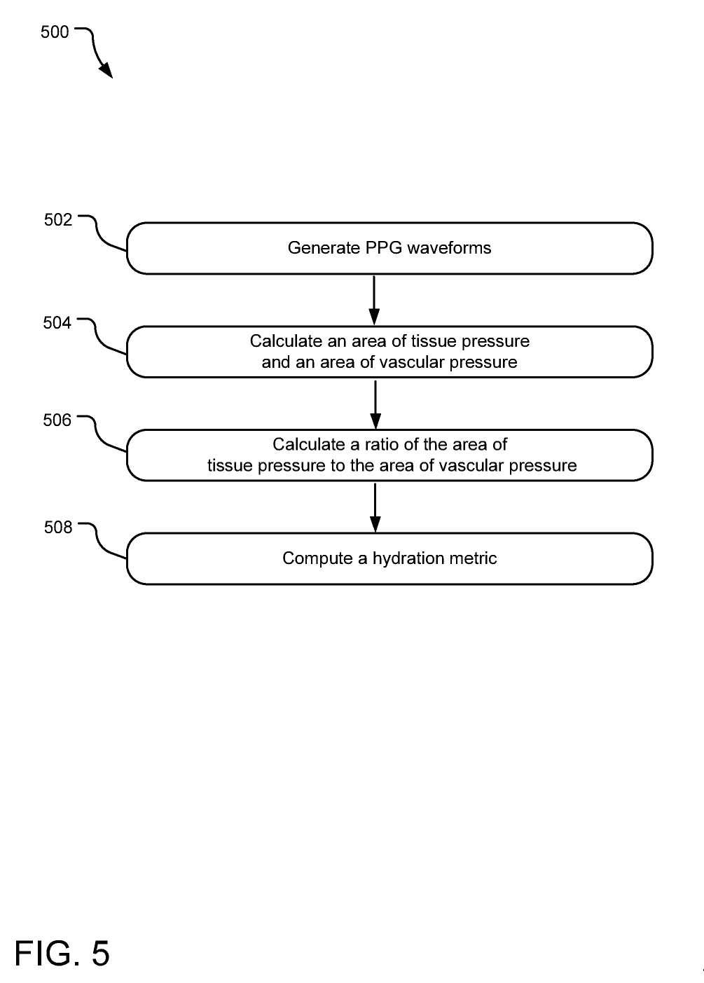

In another implementation, there can be a plurality of light sources 108 and a plurality of light sensors 104 configured in an array, as shown in FIG. 1c. There may be an array of light sensors 104 and light sources 108 (e.g., LEDs), which can be configured to rest on or next to the skin surface around the wrist. The array may be configured to select an optimal pairing of the light sensors and light sources that provides the best representation of the PPG waveform (described in more detail in FIG. 5).

In another implementation (not shown), there may be a plurality of LEDs, wherein one LED may be a light source and another LED may be a sensor. In another implementation (not shown), the light sensor 104 may be a near infrared spectrometer and the light source 108 may provide light in the near infrared wavelength. In another implementation (not shown), where there is sufficient ambient light, the hydration monitoring system consists of using only a photodetector or other optical sensor.

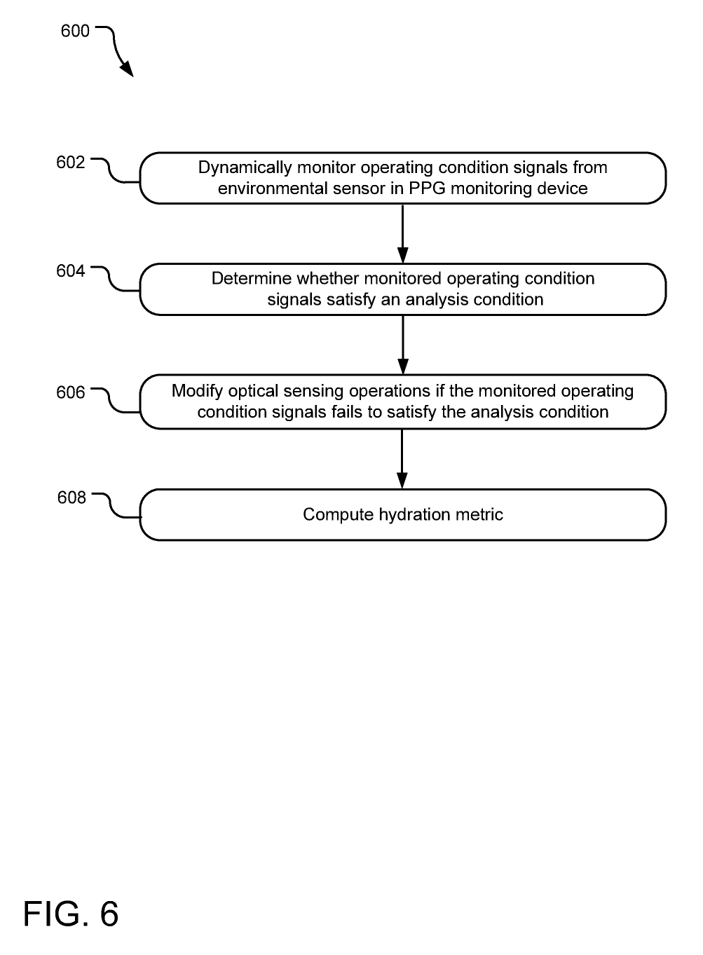

In FIG. 2, another example hydration monitoring system 200 is shown. In this implementation, environmental sensors (e.g., electrodes or conductive ground pins 210) are located on the interior of the wristlet 202 and configured to be in contact with the surface of a user's skin. The ground pins 210 measure impedance or resistance. As discussed below in FIG. 6, the hydration monitoring system 200 can monitor for skin contact integrity and surface moisture. If there is inadequate skin contact, system modifications can be made. For example, an alarm may signal the user that there is inadequate contact, and the user can readjust the fitting of the wristlet 202.

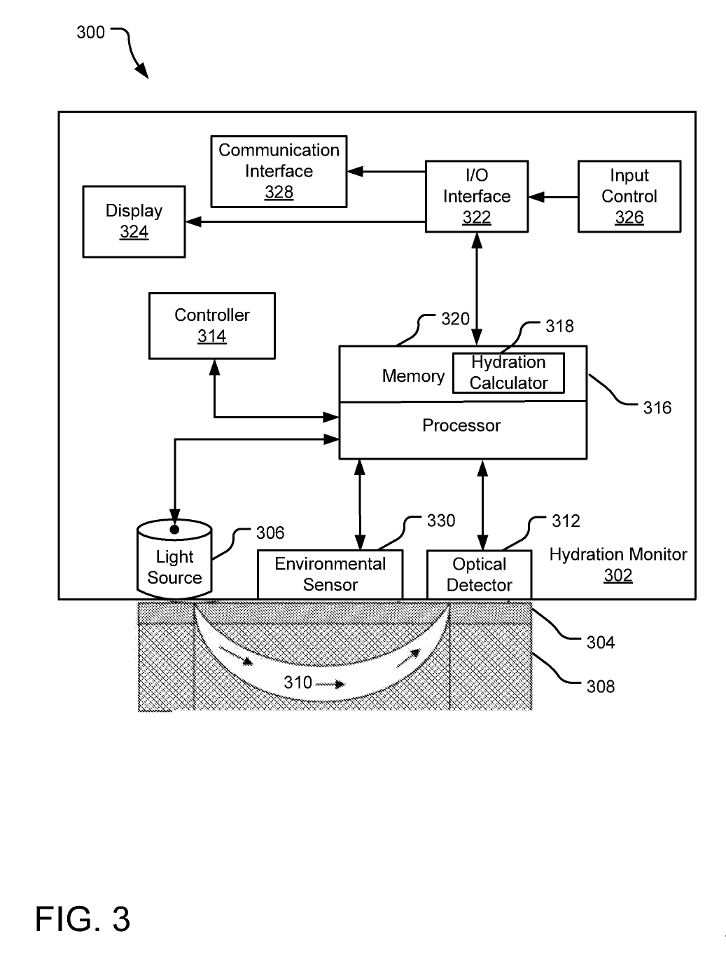

FIG. 3 shows a block diagram of an example hydration monitoring system circuitry 300 that is configured to acquire and measure a PPG waveform and determine a hydration metric representative of hydration levels in the body, which can be revealed on a display connected to the monitor. As shown in FIG. 3, the processor performs these operations in one hydration monitor 302. However, in other implementations, the PPG waveform may be obtained from an external source and measured for computation of the hydration metric in a hydration monitoring system circuitry 300.

In the hydration monitoring system circuitry 300 in FIG. 3, a hydration monitoring circuitry operates to monitor hydration when a user places a hydration monitor 302 against external skin 304 (e.g., on a user's wrist). A controller 314 sends signals to a processor 316 to activate a light source (e.g., LED) 306. The light source 306 generates light 310 against a skin 304. The light 310 is reflected through the skin 304, through a tissue 308 and through the skin 304 again for collection by an optical detector or light sensor 312.

The light sensor 312 detects the PPG waveform as a varying voltage or current level that varies with time. The relationship of the varying voltage (or current level) of the PPG waveform may be dependent on time, and can be defined as a function, or as a relationship between two variables (voltage amplitude and time) such that to each value of the independent variable (time) there corresponds a value of the dependent variable (voltage amplitude).

The processor 316 operates as a hydration metric monitoring processor and determines changes in tissue hydration levels based on the detected changes in light 310. The processor 316 interpolates PPG pulse data samples, from the light sensor 312. The processor 316 measures the PPG pulse data samples and computes tissue pressure areas and vessel pressure areas, indicative of changes in tissue volume and changes in vascular volume, respectively. A hydration calculator 318 is stored in a memory 320 in the processor 316. The hydration calculator computes a ratio of the tissue pressure area to the vessel pressure area to obtain a hydration metric or other output representative of hydration level. In another implementation, a ratio of the area of pulsatile pressure of the vascular volume is divided by the area of pulsatile pressure of the tissue volume, and/or with multipliers or constants, as provided above. The hydration metric or other output value from the hydration calculator 318 may be input into an input/output (I/O) interface 322. The I/O interface 322 is connected to one or more user-interface devices (e.g., a display unit 324) and a communications interface 328.

In one implementation, the hydration metric can be displayed on a user-interface device or display unit 324. In another implementation, the hydration metric or other output value may be communicated to the communications interface 328 for purposes of sending a signal or alarm to the user via a device, a monitoring service, a computer, wirelessly, or via a medical monitoring unit. For example, if there is an output value indicating dehydration in a patient, a communications interface 328 may signal an alarm to the patient or medical staff via a device or medical monitoring unit.

The processor can process the PPG pulse data through various algorithms and transforms (e.g., FIR filter, IIR filter, first derivative, second derivative, Fast Fourier Transform (FFT), etc.). As an example, the initial data can be analyzed with an FFT and a secondary analysis can determine whether characteristic power shifts have occurred that are correlated to a change in hydration, heart rate, etc.

The system 300 can also include one or more environmental sensors 330 (e.g., a light sensor, a temperature monitor, an accelerometer, an electrode, a gyroscope, etc.) that operatively communicate with a processor 316. In one implementation, an environmental sensor may be a temperature monitor configured to monitor the temperature of the tissue. Knowledge of the temperature of the tissue can be used to provide more accurate measurement of tissue hydration. For example, the detected temperature may be used to calculate compensation for the temperature effect on hydration. In another implementation, an environmental sensor may detect surface contact with a light sensor or a light source for analysis and selection of optimal conditions and optimal data.

An input control 326 may also be connected to the I/O 322. The input control 326 may be a button, a pressure sensor, an RF sensor, or even a touch screen. Various information may be input into the input control 326. For example, if a certain dynamic profile analysis is desired, a user may input such a request. In another example, a user may input a target hydration level into the input control 326. If a user inputs a minimum target hydration level, an alarm may be activated once a minimum value is reached, and a user may be notified visually or audibly by the monitor or another device connected directly or wirelessly. If a user inputs a maximum target hydration level, for example, a professional athlete conditioning their body for a target hydration level, a similar notification will occur. In yet another example, if a user wants to measure hydration for certain time periods or temperatures, an input control 326 could be used for such purpose. In some implementations, the operation blocks of the system 300 may be connected by a radio transmitter.

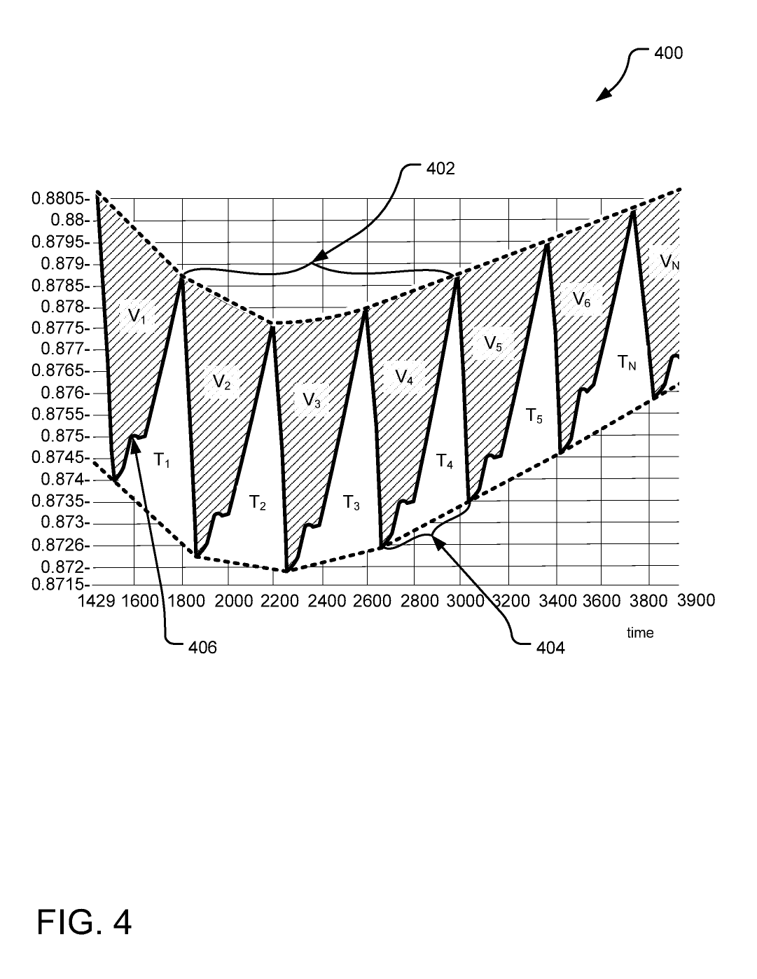

Referring to FIG. 4, an example plethysmograph 400 (measured in amplitude/time) in a hydration monitoring system graphically depicts a PPG waveform obtainable with the disclosed technology. As depicted graphically, when the heart contracts, pressure rises rapidly in the ventricle at the beginning of systole (beginning at approximately 0.8805) and soon exceeds that in the aorta. The aortic valve opens, blood is ejected, and aortic pressure rises. During the early part of the ejection, ventricular pressure exceeds aortic pressure. About halfway through ejection, the two pressures are the same and an adverse pressure gradient faces the heart (at approximately 0.874). The flow and pressure start to fall causing a “notch” in the aortic pressure wave (the dicrotic notch, shown in FIG. 4 as a dicrotic notch 406), also known as a reflected wave from the initial heart pulsatile wave. The dicrotic notch 406 marks the closure of the aortic valve. Thereafter, the ventricular pressure falls very rapidly as the heart muscle relaxes. The aortic pressure falls more slowly, with the aorta serving as a reservoir.

For illustrative purposes, the aorta may be considered as an elastic vessel or chamber and the peripheral blood vessels are considered as rigid tubes of constant resistance. For the elastic chamber (aorta), its change of volume is assumed to be absorbed by the compliance of the aortic walls as the aortic pressure increases. This elastic compliance of the aortic wall tends to smooth out the impulse of pressure the heart creates. Hence, the pressure wave as detected as a PPG waveform takes its characteristic shape.

The arterial branches that occur between the heart and the peripheral sensing site create reflection waves that also affect the shape of the PPG wave. The volume of blood has a direct effect on the PPG waveform as well as an effect on the peripheral and central nervous system, which responds in a way that affects the vessel compliance. This vessel compliance change is also reflected in the shape of the PPG wave. However, the simplifying assumption that the peripheral blood vessels are rigid tubes of constant resistance can be modified to encompass the changes that occur when tissue hydration is varying.

As overall tissue hydration increases, the compliance of the vessels, both centrally and peripherally, is reduced. This systemic reduction in vascular compliance due to systemic variance in tissue hydration can be detected as a shift in the shape of the PPG wave. The shift in shape of the PPG waveform may be detected in a way that is indicative of the relative change in tissue hydration level.

Prior to computation of a hydration metric, a PPG waveform data sample may be selected and/or filtered by monitoring profiles based on one or more sensed operating contexts (e.g., an environmental condition, a sensed activity, or a physiological condition) sensed by an environmental sensor or one or more non-sensed operating contexts (e.g., demographic inputs). The monitoring profiles can select a data sample based on parameters in the monitoring profiles, including data sample satisfaction of data integrity or result integrity. The monitoring profiles are subject to change as operating contexts change. Further, computations (e.g., the ratio of changes of tissue volume and changes of vascular volume) are subject to change depending on a change in operating contexts and monitoring profiles.

In the selected PPG waveform, the locations and amplitudes of the local peaks of the PPG waveform are identified. Several methods may be used to find the minimum points and the maximum points of the PPG waveform. In one implementation, a method of a first-derivative test to locate the relative minimum and relative maximum points may be used on the PPG function. As shown in FIG. 4, the minimum points and the maximum points are traced within triangular-shaped tracing.

When the locations (“locs”) of the minimum points and the maximum points of the PPG waveform are identified, the heart rate may also be calculated using the following equation (in MatLab script):

$$HeartRate = ( \frac{100}{mean ( diff ( locs ) )} ) \cdot 60$$

In this equation, a value of 100 is used because a sample rate may be set at 100 samples per second. The term “diff(locs)” refers to the distance between each adjacent location. The mean of the distances is determined by “mean(diff(locs)) and the fraction is multiplied by 60 to convert the dimension from inverse seconds to “per minute.” The unit of the calculated heart rate is in beats per minute (bpm).

Once the locations and the amplitudes of the minimum points and the maximum points of the PPG waveform are identified, any two adjacent minimum points (or maximum points) serve to define a line connecting the two adjacent minimum points (or maximum points), which can be calculated using line equations.

In the PPG waveform orientation shown in FIG. 4, a line 402 connects the local maximum points represent the diastolic pressure of the test subject. A line 404 connects the local minimum points represent the systolic pressure of the test subject. It is very common in the medical field to invert the PPG waveform prior to displaying it. Many medical devices that display the PPG waveform inverted the waveform so that the blood pressure is increasing in the graph when the PPG curve is shown going up. This disclosure includes either orientation of the PPG waveform.

Using the lines 402 and 404, the areas between the curves in the PPG waveform can be defined. The area between the PPG curve and the diastolic curve may be defined as the “Vessel Pressure Area” or “VPA.” The VPA is filled with lines and is labeled V1, V2, V3, . . . , VN. The area between the systolic curve and the PPG curve is defined as the “Tissue Pressure Area” or “TPA.” The TPA is not filled with lines and is labeled T1, T2, T3, . . . , TN.

Several methods of calculating the area of a region between two curves may be used. In some implementations, the application of definite integrals from the area of regions under two different curves may be used. The process of calculating the area of a region between the two different curves or functions is to subtract the function with the lesser-valued area from the function with the greater valued area. This calculation then results in the calculated area between the two curves or functions. In another implementation, one function may be subtracted from the other prior to the process of integration.

Several methods of analyzing a definite integral by partitioning the area under a curve into sub-regions may also be used. The sub-regions are approximated by rectangles of know dimension so the areas of all the rectangles can be summated to approximate the area of the definite integral. If trapezoids are used instead of rectangles, the approximation is more accurate. The digitization of an analog biometric signal may be useful for this type of trapezoidal integration. An example of trapezoidal integration use in the hydration metric MatLab script that provides the area between the TPA and the VPA is calculated with the following equations:

TPA=trapz(PlethWave)−trapz(slocs,−spks)

VPA=trapz(dlocs,dpks)−trapz(PlethWave)

After a TPA and the VPA are derived from the PPG waveform, a hydration metric is derived correlating to a ratio of the TPA divided by the VPA (or correlating to a ratio of the VPA divided by the TPA, and/or with multipliers or constants, as provided above).

FIG. 5 illustrates example operations 500 for determining a hydration metric. Operation 502 measures raw PPG pulse data samples as a sequence of data samples from a light sensor in a hydration monitoring device in an operation 502. The area of pulsatile pressure of a tissue volume and the area of pulsatile pressure of a vascular volume can be calculated in a calculating operation 504. In a calculating operation 506, a ratio of the area of pulsatile pressure of the tissue volume divided by the area of pulsatile pressure of the vascular volume. In another implementation, a ratio of the area of pulsatile pressure of the vascular volume is divided by the area of pulsatile pressure of the tissue volume, and/or with multipliers or constants, as provided above. As a result, hydration metric data results are derived.

In one implementation, the hydration metric data results may be used to refine non-invasive blood pressure calculations. Obtaining accurate measurements of arterial blood pressure by non-invasive methods (in the periphery) can be challenging because volume and flow changes may not be linearly correlated with arterial pressure. It is desirable to transform the peripheral volume signal to arterial pressure. Because hydration changes compliance of the vasculature, identifying a hydration metric by the methods disclosed herein can refine non-invasive blood pressure calculations to account for change in vasculature compliance. For example, the pulse interval between an EKG signal and the pressure pulse at an extremity can be more accurately analyzed.

In FIG. 6, examples operations 600 for autoconfiguration of a hydration monitoring device are shown. The hydration monitoring device can dynamically monitor operating condition signals from an environmental sensor in the hydration monitoring device in a monitoring operation 602. By monitoring hydration monitoring device operating conditions (e.g., environmental conditions, surface contact, temperature, system parameters, light output), the hydration monitoring device can determine whether the monitored operating condition signals satisfy an analysis condition in a determining operation 604. For example, data or components (e.g., light sources or sensors) may be analyzed with an adaptive ability to optimize operating components based on input or output. Monitoring can include analyzing signal strength, excessive noise, LED output for possible adjustment, evaluating sensor gain to compensate for changes in ambient light, or reviewing heart rate, temperature, and/or accelerometer readings.

If the monitored operating condition signals do not satisfy an analysis condition in the determining operation 604, then the hydration monitoring device can autoconfigure or modify optical sensing operations in the hydration monitoring device in a modifying operation 604.

For example, in one implementation, during the monitoring operation 602, the hydration monitoring device dynamically autodetects operating condition signals for the best output. The hydration monitoring device can determine whether the operating condition signals satisfy a condition of the best operating condition signal output in a determining operation 604. Then, the hydration monitoring device can select use of at least one of a plurality of sensors and/or lights sources producing the best output, collect the output from those sensors and/or lights sources only, and discard bad output or noise in the modifying operation 606.

In another implementation, a hydration monitoring device with multiple sensors may detect where on a user's arm the operating condition signal from a certain sensor and/or lights source produces the best output, and stop using the other sensors and/or lights sources, or discard PPG pulse data samples received from the other sensors and/or lights sources, or, as a function of power management, enable the other sensors and/or lights sources to enter a lower energy mode (e.g., turn the poorly sensing sensors off, or the enter a sleep mode).

In another implementation, an array of LEDs strobed and selected may be positioned at the back of the hydration monitoring device (e.g., wristlet). The operating condition signals monitored are greatly improved if the LEDs are arranged in an array protruding from a hydration monitoring device against the surface of a user's skin.

In another implementation, photo detectors may be located around a wristlet and the photodetector selected and used is the one that has the best light signal. This approach can use optical absorption of a variant vasculature in a dynamic sensor array.

Depending on a specific light requirement, the hydration monitoring device can monitor operating condition signals specific to the amount of light transmitted in the hydration monitoring device. For example, an LED may be electronically selectable as a light source or a light sensor, automatically or manually. In another implementation, where there is an array of light sensors and/or light sources (e.g., LEDs) capable of providing light, the hydration monitoring device dynamically monitors the light output of each light sensor or light source and controls use, operation, and data collection dependent on output.

In another example, a hydration monitoring device using ambient light as a light source may require supplemental light from another source, such as a LED. The hydration monitoring device in this example can detect the need for supplemental light and select the LED for back-up. A sawtooth wave, a non-sinusoidal waveform, can be applied as an LED drive current in the hydration monitoring system. The LED drive current amplitude ramps upward when ambient light is insufficient until the composite light becomes sufficient. If the ambient light becomes insufficient, for example, when a user walks towards a dark area, then the sensed waveform sharply drops. Upon detection of the drop in light power, the hydration monitoring device can activate a back-up or alternative light source.

In another implementation, the system can monitor for adequate sensor contact. There may be at least two electrodes (e.g., conductive ground pins) in contact with the surface of the skin that measure impedance or resistance. The ground pins monitor for skin contact integrity and surface moisture. For example, as a user wears a hydration monitoring device (e.g., wristlet) to monitor hydration during exercise, perspiration may present between the sensors on the wristlet and the user's skin. The wristlet detects the surface moisture from operating condition signals and modifies the optical sensing operations by using components (those unaffected by the surface moisture) to obtain data or send alerts of inadequate readings via an alarm.

For example, in one implementation, alarms may be implemented in the monitoring operation 602 to communicate via a communications interface if the optical sensing operations cannot be modified. For example, if a hydration monitoring device determines overhydration or dehydration, the optical sensing operations may not be modified to overcome the failure to satisfy an analysis condition. Therefore, alarms may signal wearables, water sources, and other appliances to notify a user, healthcare provider, or other person or system. Such conditions can be tailored to when a user is at rest and/or during a certain activity. In another implementation, profiles could be selected based on different temperatures monitored with a temperature sensor, and activate an alarm based on the selections.

In another implementation, the hydration monitoring device can monitor power usage. If the hydration monitoring device receives sufficient power during operations, the hydration monitoring device may change to a lower power setting or power off. For example, if there is sufficient light from ambient light, LEDs in the hydration monitoring device may turn off. Or, if one LED is providing optimal use evidenced by optimal data output, other LEDs in the system may enter a lower power mode by reducing the light production or powering off. On the other hand, if poor data output is determined from operation condition signals, the hydration monitoring system can increase LED output or activate a battery to obtain more reliable PPG pulse data.

After the hydration monitoring system modifies the optical sensing operations in the modifying operation 606, the system can compute a biometric (e.g, hydration metric) per the method disclosed in FIG. 5, by acquiring PPG pulse data samples produced in the hydration monitoring device that satisfies analysis conditions.

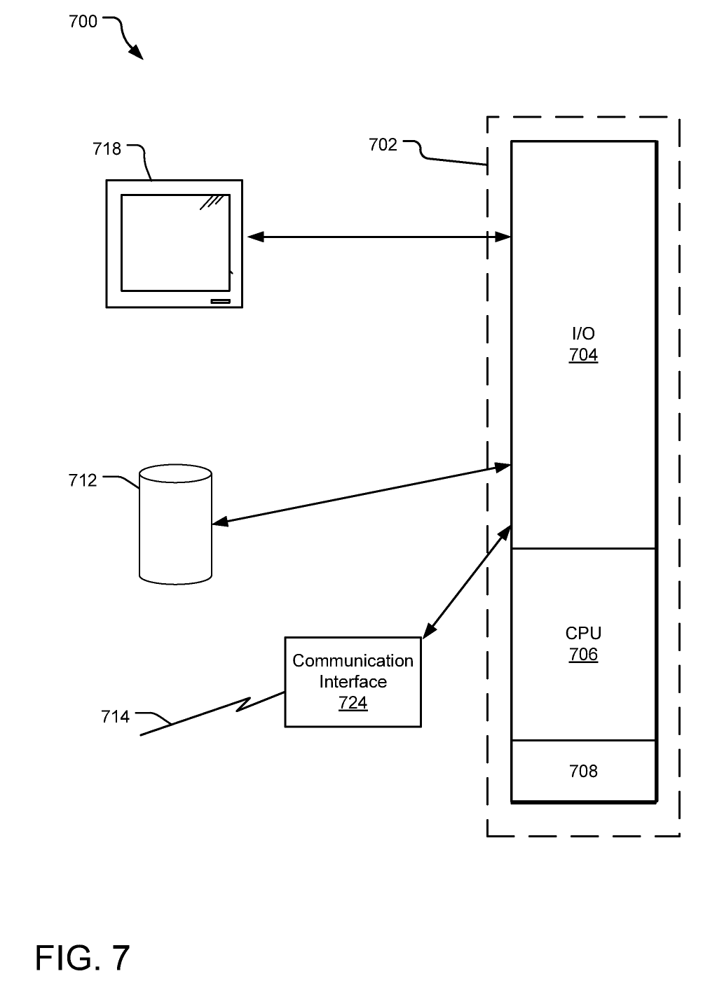

Referring to FIG. 7, a block diagram of a computer system 700 suitable for implementing one or more aspects of a system for receiving and analyzing PPG pulse data and determining a hydration metric is shown. The computer system 700 is capable of executing a computer program product embodied in a tangible computer-readable storage medium to execute a computer process. Data and program files may be input to the computer system 700, which reads the files and executes the programs therein using one or more processors. Some of the elements of a computer system 700 are shown in FIG. 7 wherein a processor 702 is shown having an input/output (I/O) section 704, a Central Processing Unit (CPU) 706, and a memory section 708. There may be one or more processors 702, such that the processor 702 of the computing system 700 comprises a single central-processing unit 706, or a plurality of processing units. The processors may be single core or multi-core processors. The computing system 700 may be a conventional computer, a distributed computer, or any other type of computer. The described technology is optionally implemented in software loaded in memory 708, a disc storage unit 712, and/or communicated via a wired or wireless network link 714 on a carrier signal (e.g., Ethernet, 3G wireless, 5G wireless, LTE (Long Term Evolution)) thereby transforming the computing system 700 in FIG. 7 to a special purpose machine for implementing the described operations.

The I/O section 704 may be connected to one or more user-interface devices (e.g., a keyboard, a touch-screen display unit 718, etc.) or a disc storage unit 712. Computer program products containing mechanisms to effectuate the systems and methods in accordance with the described technology may reside in the memory section 704 or on the storage unit 712 of such a system 700.

A communication interface 724 is capable of connecting the computer system 700 to an enterprise network via the network link 714, through which the computer system can receive instructions and data embodied in a carrier wave. When used in a local area networking (LAN) environment, the computing system 700 is connected (by wired connection or wirelessly) to a local network through the communication interface 724, which is one type of communications device. When used in a wide-area-networking (WAN) environment, the computing system 700 typically includes a modem, a network adapter, or any other type of communications device for establishing communications over the wide area network. In a networked environment, program modules depicted relative to the computing system 700 or portions thereof, may be stored in a remote memory storage device. It is appreciated that the network connections shown are examples of communications devices for and other means of establishing a communications link between the computers may be used.

In an example implementation, a user interface software module, a communication interface, an input/output interface module and other modules may be embodied by instructions stored in memory 708 and/or the storage unit 712 and executed by the processor 702. Further, local computing systems, remote data sources and/or services, and other associated logic represent firmware, hardware, and/or software, which may be configured to assist in obtaining hydration measurements. A hydration monitoring system may be implemented using a general purpose computer and specialized software (such as a server executing service software), a special purpose computing system and specialized software (such as a mobile device or network appliance executing service software), or other computing configurations. In addition, PPG pulse data samples, hydration metric data results, and system optimization parameters may be stored in the memory 708 and/or the storage unit 712 and executed by the processor 702.

It should be understood that the hydration monitoring system may be implemented in software executing on a stand-alone computer system, whether connected to a hydration monitor device or not. In yet another implementation, the hydration monitoring system may be integrated into a device (e.g., a wristlet).

The implementations of the invention described herein are implemented as logical steps in one or more computer systems. The logical operations of the present invention are implemented (1) as a sequence of processor-implemented steps executed in one or more computer systems and (2) as interconnected machine or circuit modules within one or more computer systems. The implementation is a matter of choice, dependent on the performance requirements of the computer system implementing the invention. Accordingly, the logical operations making up the implementations of the invention described herein are referred to variously as operations, steps, objects, or modules. Furthermore, it should be understood that logical operations may be performed in any order, adding and omitting as desired, unless explicitly claimed otherwise or a specific order is inherently necessitated by the claim language.

Data storage and/or memory may be embodied by various types of storage, such as hard disk media, a storage array containing multiple storage devices, optical media, solid-state drive technology, ROM, RAM, and other technology. The operations may be implemented in firmware, software, hard-wired circuitry, gate array technology and other technologies, whether executed or assisted by a microprocessor, a microprocessor core, a microcontroller, special purpose circuitry, or other processing technologies. It should be understood that a write controller, a storage controller, data write circuitry, data read and recovery circuitry, a sorting module, and other functional modules of a data storage system may include or work in concert with a processor for processing processor-readable instructions for performing a system-implemented process.

For purposes of this description and meaning of the claims, the term “memory” (e.g., memory 320, memory 708) means a tangible data storage device, including non-volatile memories (such as flash memory and the like) and volatile memories (such as dynamic random access memory and the like). The computer instructions either permanently or temporarily reside in the memory, along with other information such as data, virtual mappings, operating systems, applications, and the like that are accessed by a computer processor to perform the desired functionality. The term “memory” expressly does not include a transitory medium such as a carrier signal, but the computer instructions can be transferred to the memory wirelessly.

The above specification, examples, and data provide a complete description of the structure and use of exemplary implementations of the invention. Since many implementations of the invention can be made without departing from the spirit and scope of the invention, the invention resides in the claims hereinafter appended. Furthermore, structural features of the different implementations may be combined in yet another implementation without departing from the recited claims.

What is claimed is:

1. A method comprising:
determining whether monitored operating condition signals associated with a photoplethysmographic (PPG) monitoring device satisfy an analysis condition based on the monitored operating condition signals from an environmental sensor in the PPG monitoring device;
modifying optical sensing operations in the PPG monitoring device if the monitored operating condition signals fail to satisfy the analysis condition; and
measuring changes in tissue volume within body tissue of a subject based on the modified optical sensing operations, the modified optical sensing operations satisfying the analysis condition.

2. The method of claim 1, further comprising measuring changes in vascular volume within body tissue of a subject based on the modified optical sensing operations, the modified optical sensing operations satisfying the analysis condition.

3. The method of claim 2, further comprising computing a hydration metric based on data measured by the PPG monitoring device representing changes in tissue volume and changes in vascular volume within body tissue of a subject acquired from the modified optical sensing operations.

4. The method of claim 3, further comprising communicating the computed hydration metric of the body tissue of the subject via a communications interface.

5. The method of claim 1, wherein the environmental sensor includes at least one of a light sensor, a temperature monitor, an accelerometer, or an electrode.

6. The method of claim 1, further comprising monitoring operating condition signals associated with a photoplethysmographic (PPG) monitoring device from an environmental sensor in the PPG monitoring device.

7. The method of claim 1, wherein the monitoring operating condition signals operation further comprises monitoring light received through the body tissue of the subject.

8. The method of claim 1, wherein the monitoring operating condition signals operation further comprises monitoring temperature of the body tissue of the subject.

9. The method of claim 1, wherein the monitoring operating condition signals operation further comprises monitoring motion of the monitoring device.

10. The method of claim 1, wherein the monitoring operating condition signals operation further comprises monitoring sensor contact with the body tissue of the subject.

11. The method of claim 1, wherein the monitoring operating condition signals operation is based on bioimpedance detected between at least two electrodes.

12. The method of claim 1, modifying optical sensing operations in the monitoring device if the monitored operating condition signals fail to satisfy the analysis condition further comprises selecting at least one of a plurality of sensors of the monitoring device.

13. The method of claim 1, wherein modifying optical sensing operations in the monitoring device if the monitored operating condition signals fail to satisfy the analysis condition further comprises using one of a plurality of light sources of the monitoring device.

14. The method of claim 1, modifying optical sensing operations in the monitoring device if the monitored operating condition signals fail to satisfy the analysis condition further comprises enabling at least one operating sensor to enter a lower energy mode based on data received in the determining operation.

15. The method of claim 1, modifying optical sensing operations in the monitoring device if the monitored operating condition signals fail to satisfy the analysis condition enabling at least one light source to enter a lower energy mode based on data received in the determining operation.

16. The method of claim 1, further comprising communicating an alarm via a communications interface if the optical sensing operations cannot be modified.

17. A system, comprising:
a processor configured to determine whether monitored operating condition signals associated with a photoplethysmographic (PPG) monitoring device satisfy an analysis condition based on the monitored operating condition signals from an environmental sensor in the PPG monitoring device, modify optical sensing operations in the PPG monitoring device if the monitored operating condition signals fail to satisfy the analysis condition, and measure changes in tissue volume within body tissue of a subject based on the modified optical sensing operations, the modified optical sensing operations satisfying the analysis condition; and
a communications interface configured to communicate the measured changes in tissue volume of the body tissue of the subject via a communications interface.

18. One or more tangible computer-readable storage media encoding computer-executable instructions for executing on a computer system a computer process, the computer process comprising:
determining whether monitored operating condition signals associated with a photoplethysmographic (PPG) monitoring device satisfy an analysis condition based on the monitored operating condition signals from an environmental sensor in the PPG monitoring device;
modifying optical sensing operations in the PPG monitoring device if the monitored operating condition signals fail to satisfy the analysis condition; and
measuring changes in tissue volume within body tissue of a subject based on the modified optical sensing operations, the modified optical sensing operations satisfying the analysis condition.

19. The one or more tangible computer-readable storage media of claim 18, further comprising measuring changes in vascular volume within body tissue of a subject based on the modified optical sensing operations, the modified optical sensing operations satisfying the analysis condition.

20. The one or more tangible computer-readable storage media of claim 19, further comprising computing a hydration metric based on data measured by the PPG monitoring device representing changes in tissue volume and changes in vascular volume within body tissue of a subject acquired from the modified optical sensing operations.

21. The one or more tangible computer-readable storage media of claim 20, further comprising communicating the computed hydration metric of the body tissue of the subject via a communications interface.

22. The one or more tangible computer-readable storage media of claim 18, wherein the environmental sensor includes at least one of a light sensor, a temperature monitor, an accelerometer, or an electrode.

23. The one or more tangible computer-readable storage media of claim 18, further comprising monitoring operating condition signals associated with a photoplethysmographic (PPG) monitoring device from an environmental sensor in the PPG monitoring device.

24. The one or more tangible computer-readable storage media of claim 23, wherein the monitoring operating condition signals operation further comprises monitoring light received through the body tissue of the subject.

25. The one or more tangible computer-readable storage media of claim 23, wherein the monitoring operating condition signals operation further comprises monitoring temperature of the body tissue of the subject.

26. The one or more tangible computer-readable storage media of claim 23, wherein the monitoring operating condition signals operation further comprises monitoring motion of the monitoring device.

27. The one or more tangible computer-readable storage media of claim 23, wherein the monitoring operating condition signals operation further comprises monitoring sensor contact with the body tissue of the subject.

28. The one or more tangible computer-readable storage media of claim 23, wherein the monitoring operating condition signals operation is based on bioimpedance detected between at least two electrodes.

29. The one or more tangible computer-readable storage media of claim 18, wherein modifying optical sensing operations in the monitoring device if the monitored operating condition signals fail to satisfy the analysis condition further comprises selecting at least one of a plurality of sensors of the monitoring device.

30. The one or more tangible computer-readable storage media of claim 18, wherein modifying optical sensing operations in the monitoring device if the monitored operating condition signals fail to satisfy the analysis condition further comprises using one of a plurality of light sources of the monitoring device.

31. The one or more tangible computer-readable storage media of claim 18, wherein modifying optical sensing operations in the monitoring device if the monitored operating condition signals fail to satisfy the analysis condition further comprises enabling at least one operating sensor to enter a lower energy mode based on data received in the determining operation.

32. The one or more tangible computer-readable storage media of claim 18, wherein modifying optical sensing operations in the monitoring device if the monitored operating condition signals fail to satisfy the analysis condition further comprises enabling at least one light source to enter a lower energy mode based on data received in the determining operation.

33. The one or more tangible computer-readable storage media of claim 18, further comprising communicating an alarm via a communications interface if the optical sensing operations cannot be modified.
